(19) United States
(12) Patent Application Publication
(10) Pub. No.: US 2025/0287923 A1
(43) Pub. Date: Sep. 18, 2025

(54) Dual-Wavelength Phototherapy Animal Health System

(71) Applicant: Kian Lawrence, Long Beach, CA (US)

(72) Inventor: Kian Lawrence, Long Beach, CA (US)

(21) Appl. No.: 19/057,577

(22) Filed: Feb. 19, 2025

Related U.S. Application Data
(60) Provisional application No. 63/557,487, filed on Feb. 24, 2024.

(51) Int. Cl. A01K 15/02; A61H 13/00; A61H 23/02; A61N 5/06

(52) U.S. Cl. CPC A01K 15/026; A61H 13/00; A61H 23/0254; A61N 5/0603; A61N 5/0624; A61H 2203/03; A61N 2005/0606; A61N 2005/0626; A61N 2005/0651; A61N 2005/0663; A61N 2005/0668

(57) ABSTRACT

A spherical, bone or other shaped pet health device includes a 0.5% to 5% thermoplastic resin casing, equipped with a dual wavelength LED system emitting blue (400-460 nm) and red (600-670 nm) light. The device incorporates a printed circuit board (PCB) with LEDs, a voltage regulator, and a motion sensor which activates the LEDs upon impact, with light emission lasting between 4-6 minutes. Some embodiments include a capacitive touch sensor or pressure sensor to trigger the LED system. The device may be charged via USB cable or completely sealed and charged only via induction or have a removable water resistant inner compartment with removable batteries. The outer can feature texture, scent infusion and indentations for flavor agents and health supplements such as probiotics. Additional embodiments may include a vibration motor and conductive fillers in the casing to enhance signal transmission for various health promoting applications.

### BACKGROUND OF THE INVENTION

Owners of domesticated animals face significant challenges in maintaining their pets' oral health, primarily due to the difficulty of current solutions such as brushing, the high cost and risks of professional cleanings, and the limitations of existing oral care products. Many dogs and cats resist traditional toothbrushes, making routine cleaning stressful and ineffective, while alternative solutions such as dental chews and water additives often fail to remove plaque and tartar. As a result, periodontal disease is prevalent, affecting approximately 80% of dogs by age three, leading to chronic bad breath, pain, tooth loss, and systemic health risks, including heart, liver, and kidney disease. Given these challenges, there is a widespread need for a more efficient, non-invasive, and stress-free solution to canine and feline oral hygiene that simultaneously reduces plaque buildup and remedies the harmful effects of the bacteria responsible for periodontal disease.

### BRIEF DESCRIPTION

The spherical device has a substantially firm thermoplastic resin casing. Some embodiments of the device are spherical and have a diameter of as low as 50 millimeters, while others have a diameter of up to 130 millimeters. Some embodiments resemble the shape of a bone, stick, or a star polyhedron. Some embodiments of the device contain the same light emission system, embedded into a prey-shaped casing wired to a power source in the handle of a rod, similar to a lined fishing rod; this embodiment is paired with an attractant such as catnip, feathers, or beef tallow. Some embodiments of the device include a designated section for inserting a threaded cylindrical component made of thermoplastic resin, which houses a battery-powered printed circuit board (PCB) with at least two attached light-emitting diodes, as well as a voltage regulator. Some embodiments of the device have a completely-sealed, water-resistant, inner compartment containing a rechargeable battery-powered PCB with at least two attached light-emitting diodes; the batteries are recharged wirelessly through inductive charging, which involves transferring energy from a primary coil (charger) to a secondary coil (embedded within the device) via electromagnetic fields. The at least two light-emitting diodes (dual wavelength system) respectively emit blue light between 400 and 460 nanometers and red light between 600 and 670 nanometers. Some embodiments of this dual-wavelength LED system are configured to emit light in a pulsing sequence. The outer layer of the device consists of thermoplastic resin. The exterior is significantly transparent, between the levels of 0.5% and 15% transparency. The surface has a ridged texture. Some embodiments include asymmetrical protrusions comprised of the same thermoplastic resin material. The surface contains indentations between 4 mm and 12 mm in diameter and between 2 mm and 8 mm deep. Some embodiments include an outer layer infused with scent. Some embodiments of the system include a complementary, synergistic, flavored, food-grade substance. Some embodiments of these food-grade substances contain probiotic bacteria. Some embodiments include a reservoir within the device that releases flavored agents in sync with light activation. The device contains a PCB that is activated by a pressure sensor set to activate upon impact, such as a light bounce or tap. The PCB within the device is set to activate LED light output for between 4 and 6 minutes. Some embodiments include a pressure sensor that activates a vibration motor within certain parameters of pressure. Some embodiments comprise a sensor designed to activate vibration and/or light emission upon detection of a signal loop being established through sensory nodules embedded into conductive plastic plates. The dual wavelength system contains at least two light-emitting diodes, emitting blue light between 400 and 460 nanometers and red light between 600 and 670 nanometers, respectively. Some embodiments include a conductive thermoplastic resin casing embedded with a conductive filler selected from graphene, silver-coated glass microspheres, or titanium dioxide with antimony-doped tin oxide, wherein an embedded conductive pathway electrically couples the casing to a capacitive touch sensor, said sensor being in electrical communication with a printed circuit board (PCB) configured to detect changes in capacitance upon contact with a conductive object, thereby activating a dual-wavelength LED system emitting blue light (400-460 nm) and red light (600-670 nm).

The inner mechanism of the device consists of a PCB with Light Emitting Diodes (LEDs) and a motion sensor and a voltage regulator. When the device is bounced, the motion sensor detects the impact and sends a signal to a microcontroller on the PCB. The microcontroller then activates the LEDs, turning them on. A timing circuit is programmed to keep the LEDs illuminated for between 4 and 6 minutes. After this duration, the timer circuit automatically turns off the LEDs. Some embodiments include a conductive thermoplastic resin casing (exterior) comprising a conductive filler providing electrical signal transmission upon contact with a conductive object (body), said conductive filler in electrical communication with a capacitive touch sensor electrically coupled to the conductive thermoplastic resin casing via an embedded conductive pathway, wherein said capacitive touch sensor is configured to detect a change in capacitance at said exterior upon contact with a conductive object; said sensor being in electrical communication with the printed circuit board. Some embodiments include a vibration motor.

### BRIEF DESCRIPTION OF DRAWINGS

FIG. 1 is an isometric view of the exterior of the device.

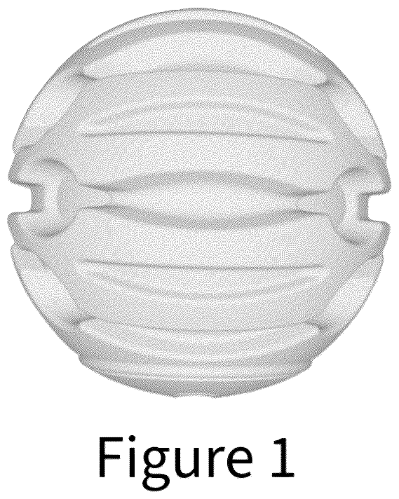

FIG. 2 is an isometric view of the exterior of the device.

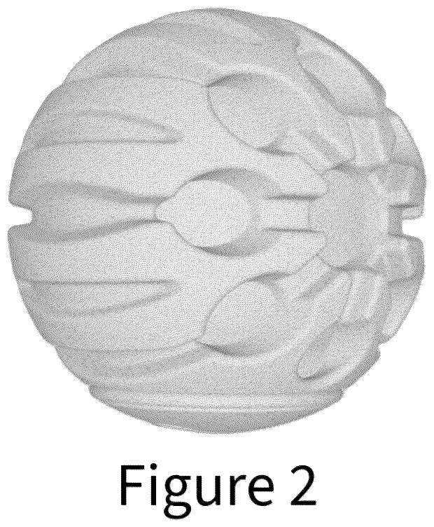

FIG. 3 is an isometric view of the exterior of the device.

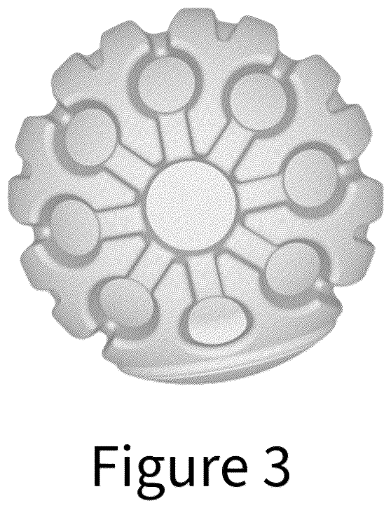

FIG. 4 is an isometric view of the exterior of the device, including the screw-in light system holder.

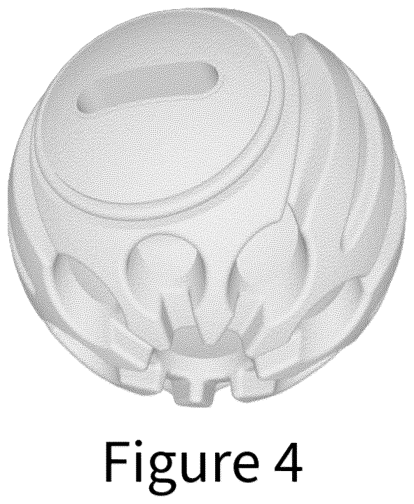

FIG. 5 depicts the printed circuit board of the device including the light-emitting diodes.

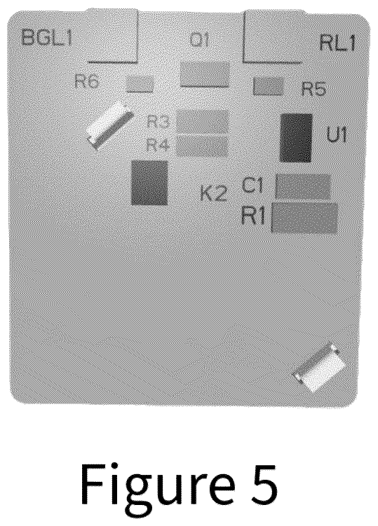

FIG. 6 depicts the cell battery holder attached to the printed circuit board.

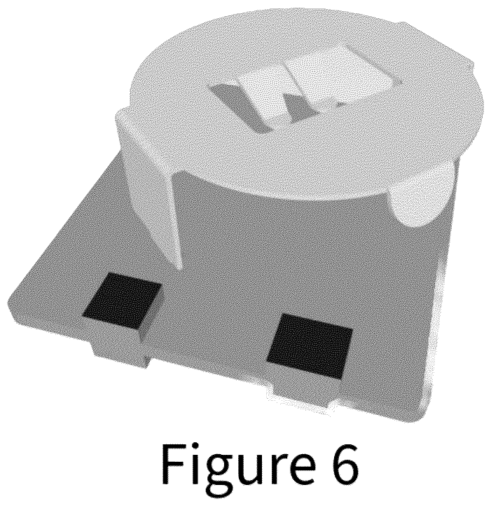

FIG. 7 depicts the cell battery holder attached to the printed circuit board.

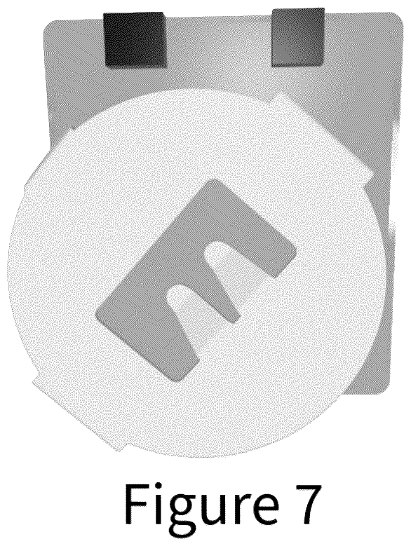

FIG. 8 depicts the screw-in light system holder with the light system inserted.

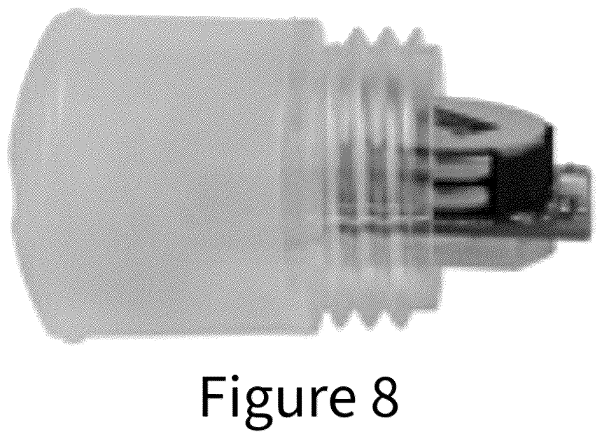

### DETAILED DESCRIPTION

Oral health is a significant and often overlooked aspect of overall health in domesticated animals, particularly dogs. Studies indicate that by the age of three, the majority of dogs exhibit some form of periodontal disease, ranging from gingivitis to severe periodontitis. These conditions, caused primarily by the buildup of plaque and tartar, can lead to inflammation, gum recession, tooth loss, and systemic health issues such as infections spreading to the heart, liver, and kidneys. The impracticality of necessary, periodic dental cleanings yields a widespread demand for the production and distribution of mechanisms enticing to domesticated animals that simultaneously clean the mouth and reduce inflammation and irritation.

The device includes multiple enticement mechanisms to ensure engagement and prolonged use by the animal. Some embodiments of the device include an outer layer infused with scent, allowing an owner of an animal to attract their animal to the device with less effort than other canine dental-health mechanisms present in the market. Some embodiments of the device comprise indentations that can hold and release food-grade flavoring substances (eg., beef tallow or peanut butter). Some embodiments of these food-grade substances contain probiotics or health supplements. The release of this substance(s) enhances the animal's interest in the device, increasing the efficiency of the mechanism.

The device has a substantially firm, water-resistant, thermoplastic resin casing (exterior) to inhibit damage to the inner mechanism. Some embodiments of said exterior are infused with enticing scent. Some embodiments of the device are spherical and have a diameter of as low as 50 millimeters, while others have a diameter of up to 130 millimeters. Some embodiments resemble a bone in shape. Some embodiments resemble a stick in shape. Some embodiments resemble a star polyhedron in shape. Some embodiments of the device contain the same light emission system, embedded into a prey-shaped casing wired to a power source in the handle of a rod, similar to a lined fishing rod; this embodiment is paired with an attractant such as catnip, feathers, or beef tallow to engage the domesticated animal and promote prolonged utilization of the device. This exterior has a transparency range between 0.5% and 15% to allow controlled light diffusion. The outer surface of the device is textured with raised ridges that mimic the tactile sensation of natural objects, such as bones or rawhide. This textured surface massages the animal's gums, scrapes plaque and tartar buildup off the teeth, and stimulates pH-balancing saliva. Some embodiments include asymmetrical protrusions comprised of the same thermoplastic resin material that serve to generate unpredictable bounce patterns upon landing, increasing the engagement of the animal. Some embodiments include a pressure sensor activated within designated parameters of external force, thus activating a vibration motor, stimulating gum tissue, and dislodging plaque and tartar. Some embodiments include a conductive thermoplastic resin casing embedded with a conductive filler selected from graphene, silver-coated glass microspheres, or titanium dioxide with antimony-doped tin oxide, wherein an embedded conductive pathway electrically couples the casing to a capacitive touch sensor, said sensor being in electrical communication with a printed circuit board (PCB) configured to detect changes in capacitance upon contact with a conductive object, thereby activating a dual-wavelength LED system emitting blue light (400-460 nm) and red light (600-670 nm).

Blue light in the range of 400 to 460 nanometers has a potent anti-pathogenic effect. The anti-pathogenic effect of this blue light arises from its interaction with endogenous porphyrins, which are naturally occurring compounds found in certain bacteria. When exposed to blue light, these porphyrins absorb the energy and undergo photodynamic activation, resulting in the production of reactive oxygen species, such as singlet oxygen and superoxide radicals. These highly reactive molecules primarily oxidize the lipids in the bacterial cell membranes of pathogenic bacteria such as Porphyromonas gingivalis, Prevotella, and Aggregatibacter actinomycetemcomitans, causing their cell lysis. Said pathogenic bacteria, among others, are responsible for the development of plaque and tartar. The dual wavelength system contains at least two light-emitting diodes, emitting blue light between 400 and 460 nanometers and red light between 600 and 670 nanometers, respectively. Some embodiments of this dual-wavelength LED system are configured to emit blue light in a pulsing sequence, emitting between 400 and 460 nm at a controlled frequency, serving to optimize the efficiency of pathogenic cell lysis, reduce heat production, and provide deeper tissue penetration than continuous blue light of the same power and wavelength.

Red light in the range of 600 to 670 nanometers plays a critical role in promoting photobiomodulation, a biological process wherein light energy triggers cellular responses that enhance regeneration. This wavelength is particularly effective due to its ability to penetrate tissues more deeply than shorter wavelengths, allowing it to reach the mitochondria of cells. This red light enhances the activity of cytochrome c oxidase, an enzyme complex within the mitochondrial electron transport chain, thus facilitating the conversion of adenosine diphosphate into adenosine triphosphate, the primary energy currency of cells. Enhanced adenosine triphosphate levels support key metabolic activities, including protein synthesis, ion transport, and cell signaling, aiding optimal collagen production, angiogenesis, and anti-inflammatory effects. Red light reduces the production of pro-inflammatory cytokines, mitigating swelling and discomfort in inflamed or injured areas. The dual wavelength system contains at least two light-emitting diodes, emitting blue light between 400 and 460 nanometers and red light between 600 and 670 nanometers, respectively. Some embodiments of this dual-wavelength LED system are configured to emit red light in a pulsing sequence, emitting between 600 and 670 nm at a controlled frequency, serving to reduce heat production and provide deeper tissue penetration than continuous red light of the same power and wavelength.

Though less susceptible to damage from reactive oxygen species than pathogenic cells due to their robust network of antioxidant systems, mammalian bodily cells can experience oxidative stress from reactive oxygen species in their vicinity, leading to inflammation or tissue sensitivity. Red light at 670 nanometers increases nitric oxide release from cells, which dilates blood vessels and increases circulation to the treated area, aiding the body's natural anti-inflammatory response. Red light at 670 nanometers reduces the production of pro-inflammatory cytokines (eg., IL-1β, IL-6, TNF-α) and increases the production of anti-inflammatory cytokines (eg., IL-4, IL-10, TGF-β), promoting a balanced immune response. Red light at 670 nanometers upregulates the production of growth factor molecules that treat inflammation such as transforming growth factor-beta and vascular endothelial growth factor, which contribute to collagen synthesis and angiogenesis, respectively. This is where the simultaneous inclusion of red light at 670 nanometers demonstrates a synergistic effect that surpasses the capabilities of using blue and red light apart. The dual wavelength system contains at least two light-emitting diodes, emitting blue light between 400 and 460 nanometers and red light between 600 and 670 nanometers, respectively.

With an optimal environment for the introduction of probiotics provided by reduced pathogenic activity, enhanced circulation, and tissue repair functions, the combination of blue and red light with a probiotic formula creates an additional synergistic effect. The device accompanies an embodiment of the provided synergistic, flavored, food-grade substance. Some embodiments of these substances contain probiotics. The substances serve to entice the animal to mouth the device. Embodiments of the substance containing probiotics demonstrate enhanced efficacy when administered concurrently with exposure to red light within the wavelength range of 600 to 670 nanometers and blue light within the wavelength range of 400 to 460 nanometers. This synergistic effect arises from a combination of biological and photochemical interactions that optimize the conditions for probiotic survival, colonization, and activity. Red light in the 600-670 nanometer range has been shown in prior research to enhance cellular metabolism by stimulating mitochondrial activity through photobiomodulation, thereby promoting favorable conditions for probiotic colonization and function. Blue light in the 400-460 nanometer range possesses antipathogenic properties, including the generation of reactive oxygen species, which selectively target pathogenic microorganisms, reducing their populations in environments such as the oral cavity while sparing or minimally impacting beneficial microbes. The concurrent absorption of red and blue light during probiotic consumption creates a transient ecological niche in which probiotic strains face reduced competition and environmental stress, thereby increasing their likelihood of survival and proliferation. Blue light-induced reactive oxygen species generation also disrupts porphyrin-dense bacterial biofilms, further contributing to the maintenance of microbial balance and enhancing the overall effectiveness of the probiotic formulation when used simultaneously.

1. A device comprising:
a printed circuit board with a power source including but not limited to batteries or inductive charging system within a water-resistant casing,
a thermoplastic resin exterior with a controlled light diffusion transparency range between about 0.5% and about 15%,
a dual-wavelength light emitting diode system comprising continuous, controlled blue light emissions of about 400 to about 460 nanometers and red light emissions of about 600 to about 670 nanometers, or
a pressure sensor in communication with the printed circuit board and activates a printed circuit board triggered by at least a force of a light tap by a user, wherein said pressure sensor trigger activates the emission of at least two diodes.

2. The device of claim 1, wherein a shape of the device chosen from the group consisting essentially of a sphere, a bone, a stick, and a star polyhedron.

3. The device of claim 1, wherein the light emission system being embedded into a prey-shaped casing and having a wired communication to said power source.

4. The device of claim 3, wherein said wiring communication aligned in a handle of a rod.

5. The device of claim 1, comprising a blue light emission between about 400 and about 460 nanometers pulsed at a controlled frequency.

6. The device of claim 5, wherein said controlled frequency adapted to optimize the efficiency of pathogenic cell lysis.

7. The device of claim 5, wherein said controlled frequency adapted to reduce heat production.

8. The device of claim 5, wherein said controlled frequency adapted to generate a deep tissue penetration than compared to a continuous blue light emission penetration.

9. The device of claim 1, comprising red light emission between about 600 and about 670 nanometers pulsed at a controlled frequency.

10. The device of claim 9, wherein said controlled frequency adapted to reduce heat production.

11. The device of claim 9, wherein said controlled frequency adapted to generate deeper tissue penetration than a continuous red light emission penetration.

12. The device of claim 1, comprising outer ridges adapted to remove tartar and plaque accumulation.

13. The device of claim 1, comprising a plurality of indentations in the outer surface between about four and about twelve nanometers in diameter and between about two and about eight nanometers deep.

14. The device of claim 13, wherein said plurality of indentations adapted to hold topical substances or foods, such as probiotic liquids or treats.

15. The device of claim 1, including an oral probiotic formula adapted to introduce beneficial bacteria concurrently to said red and blue light absorption.

16. The device of claim 15, including generating a synergistic effect.

17. The device of claim 1, comprising a pressure sensor activated within designated parameters of the external force to activate a vibration motor, stimulating gum tissue, and dislodging plaque and tartar.

18. The device of claim 1, wherein the emitting diode system output being activated between about four to about six minutes.

19. A device comprising:
a printed circuit board with a power source including but not limited to batteries or inductive charging system within a water-resistant casing,
an exterior with a controlled light diffusion transparency range between about 0.5% and about 15%,
a dual-wavelength light emitting diode system comprising continuous, controlled blue light emissions of about 400 to about 460 nanometers pulsed at a controlled frequency and red light emissions of about 600 to about 670 nanometers pulsed at a controlled frequency, or
a pressure sensor adapted to activate a vibration motor.

20. A device comprising:
a printed circuit board with a power source including but not limited to batteries or inductive charging system within a water-resistant casing,
a conductive thermoplastic resin casing (exterior) comprising a polymer matrix embedded with a conductive filler selected from the group consisting of graphene, silver-coated glass microspheres, or titanium dioxide combined with antimony-doped tin oxide; said conductive filler providing electrical signal transmission upon contact with a conductive object (body),
a capacitive touch sensor electrically coupled to the conductive thermoplastic resin casing via an embedded conductive pathway, wherein said capacitive touch sensor is configured to detect a change in capacitance at said exterior upon contact with a conductive object; said sensor being in electrical communication with a printed circuit board, or
a dual-wavelength light-emitting diode (LED) system in electrical communication with said PCB, wherein said LED system comprises:
a first emission of blue light within a wavelength range of about 400 to 460 nanometers, and
a second emission of red light within a wavelength range of about 600 to 670 nanometers;
wherein said PCB is configured to activate said LED system upon detection of a change in capacitance.
